(19) United States
(12) Patent Application Publication
(10) Pub. No.: US 2010/0209960 A1
(43) Pub. Date: Aug. 19, 2010

(54) METHOD FOR MEASURING MITOCHONDRIAL MEMBRANE POTENTIAL IN VERTEBRATE CELLS

(75) Inventors: Charles I. Rosenblum, Hillsborough, NJ (US); Aurawan Vongs, Old Bridge, NY (US); Douglas MacNeil, Westfield, NJ (US); Rebecca Mull, Royersford, PA (US)

Correspondence Address: MERCK, P O BOX 2000, RAHWAY, NJ, 07065-0907, US

(73) Assignee: MERCK SHARP & DOHME CORP., Rahway, NJ (US)

(21) Appl. No.: 12/677,166

(22) PCT Filed: Sep. 8, 2008
(86) PCT No.: PCT/US2008/010506

§ 371 Date: Mar. 9, 2010

Related U.S. Application Data
(60) Provisional application No. 60/993,252, filed on Sep. 10, 2007.

(51) Int. Cl. C12Q 1/02

(52) U.S. Cl. 435/29

(57) ABSTRACT

The present invention relates to homogenous fluorescence-based assays for measuring mitochondrial membrane potential in vertebrate cells. The assays use an inner filter such as Brilliant Black BN to quench non-specific fluorescence. The assays are particularly suited for ultra high-throughput screening for activators of mitochondrial uncoupling proteins, chemical uncouplers, compounds with mitochondrial toxicity, and compounds which stimulate mitochondrial biogenesis.

### CROSS-REFERENCE TO RELATED APPLICATIONS

This application claims the benefit of U.S. Provisional Application No. 60/993,252, filed Sep. 10, 2007, which is incorporated herein by reference in its entirety.

### FIELD OF THE INVENTION

The present invention relates to homogenous fluorescence-based assays for measuring mitochondrial membrane potential in vertebrate cells. The assays are particularly suited for ultra high-throughput screening for activators of mitochondrial uncoupling proteins, chemical uncouplers, compounds with mitochondrial toxicity, and compounds which stimulate mitochondrial biogenesis.

### BACKGROUND OF THE INVENTION

Ninety percent of cellular oxygen consumption and the vast majority of ATP production occur in the mitochondria. See Maragos et al., 2004, J. Neurochem 91:257-262. Mitochondrial uncoupling proteins (UCPs) couple mitochondrial membrane potential (respiration) and ATP synthesis. See Rousset et al., 2004, Diabetes 53:S130-S135. This coupling is inefficient resulting in a substantial portion of the energy used by a cell lost as heat. This inefficiency provides opportunities to modulate the level of coupling in the mitochondria of cells in order to modulate cellular metabolism. By making the coupling even less inefficient, i.e., necessitating an increase in energy consumption in order to fulfill cellular energy requirements, conditions such as obesity could potentially be treated.

Five known UCPSs are found in mammalian tissues. UCP1 is present in brown fat adipose tissue and has been found to be important in thermogenesis and regulation of body temperature. See Gum et al., 1998, Science 280:1369-1370. UCP2, found in many tissues, and UCP3, found predominantly in skeletal muscle, may be involved in basal thermogenesis and be important in limiting the production of reactive oxygen species. See Rousset et al., 2004, Diabetes 53:S130-S135. UCP2, in particular, has a protective role in atherosclerosis. See id. UCP4 and UCP5 (also known as BMCP1) are expressed in the brain, but little is known about their precise function.

Several assays have been developed that are capable of detecting uncoupling protein activity and/or measuring mitochondrial membrane potential. For a review, see Amacher, 2004, Curr Med Chem 12:1829-1839. U.S. Pat. No. 5,853,975 describes assays for uncoupling activity using flow cytometry to analyze fluorescent changes in cells. U.S. Pat. No. 6,323,039 describes assays using energy transfer. Such assays are limited, however, because they require the isolation or purification of the individual components of the assay and/or the use of washing steps, and are therefore inferior to homogeneous assays. Homogeneous assays readily permit the use of high throughput screening methods. Huang describes a non-homogeneous high throughput screening assay in eukaryotic cells that can be used in 96- or 384-well plates. See Huang, 2002, J Biomolec Screening 7:383-389. This assay requires several wash steps and could not be miniaturized to a format smaller than 384 wells. A homogeneous high throughput assay for mitochondrial membrane potential in permeabilized yeast cells has been described. See Farrelly et al., 2001, Anal Biochem 293:269-276. See also U.S. Patent Application Publication No. 2003/0170606.

Despite their clear advantages, no homogeneous assays suitable for high throughput screening have been developed that allow the detection of modulation of mitochondrial uncoupling activity in vertebrate cells.

Citation or identification of any reference in this section or any other section of this application shall not be construed as an indication that such reference is available as prior art to the present invention.

### SUMMARY OF THE INVENTION

The present invention provides methods for screening a test compound for the ability to modulate mitochondrial membrane potential within a vertebrate cell comprising the steps of a) incubating a vertebrate cell in the presence of a test compound; b) adding a fluorescent dye into said vertebrate cell, wherein the fluorescence of said cell in the presence of said fluorescent dye is a function of the mitochondrial membrane potential in said cell; c) adding an inner filter to said vertebrate cell to quench non-specific fluorescence; and d) detecting the fluorescence in said cell; wherein an alteration in the fluorescence in said cell in the presence of said test compound compared to the fluorescence in the absence of said test compound indicates an ability of said test compound to modulate mitochondrial membrane potential. The screening is preferably performed in a homogeneous format, i.e., with no washing steps. In preferred embodiments, the vertebrate cell is a mammalian cell. Suitable fluorescent dyes include tetramethylrhodamine, methyl ester (TMRM) and tetramethylrhodamine, ethyl ester (TMRE). The inner filter is preferably Brilliant Black BN.

In certain embodiments of the invention, the method further comprises recombinantly expressing an uncoupling protein in the vertebrate cell. The uncoupling protein is selected from the group consisting of UCP1, UCP2, UCP3, UCP4, or UCP5.

In certain embodiments, the methods are performed in parallel with multiple test compounds each of which is in a separate well on a multi-well plate. The multi-well plate may be a 384- or 1536-well plate. Preferably, the methods are performed in a high-throughput format. More preferably, the methods are performed using robotics in a high-throughput format comprising, for example, an automatic pipetting station, a robotic armature, and a robotic controller.

The methods of the invention are particularly useful for identifying compounds that are an activator of a mitochondrial uncoupling protein, for example, UCP1, UCP2, UCP3, UCP4, or UCP5, a chemical uncoupler, a compound having mitochondrial toxicity, a compound that stimulates mitochondrial biogenesis and/or a compound that improves mitochondrial function.

### BRIEF DESCRIPTION OF THE DRAWINGS

FIG. 1 shows a dose response curve for CCCP and DNP in CHO cells.

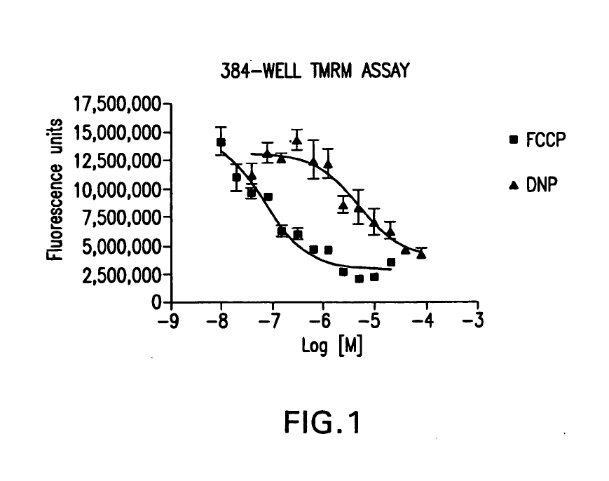

FIG. 2 shows a dose response curve for the ionophore and mitochondrial hyperpolarizer nigericin in CHO—K1 cells.

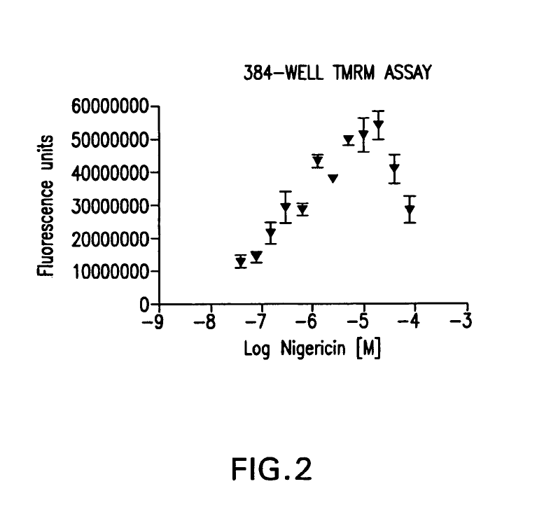

FIG. 3 shows a dose response curve for CCCP in CHO—K1 cells in a 384 well format.

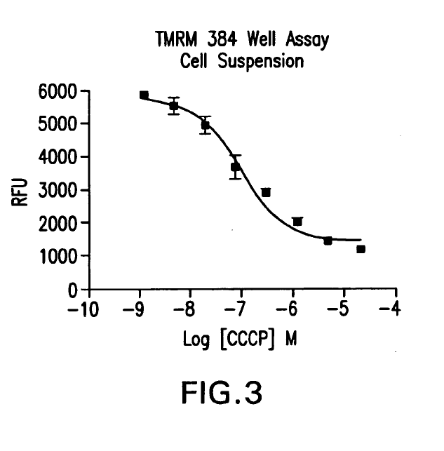

FIG. 4 shows a dose response curve for CCCP in CHO—K1 cells in a 1536 well format

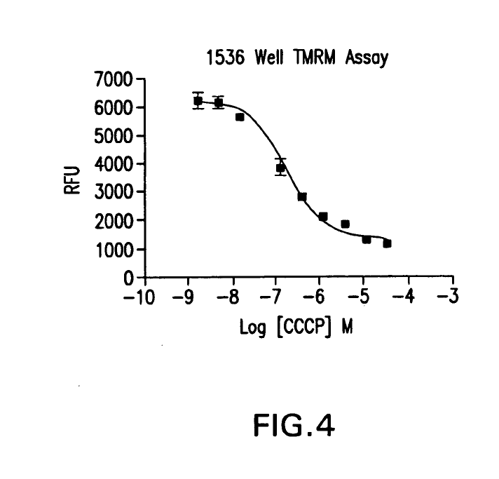

### DETAILED DESCRIPTION OF THE INVENTION

The present invention relates to a homogeneous assay for measuring mitochondrial membrane potential in vertebrate cells. By definition, a homogenous assay allows additions to be made but no removal of solution or washing of cells is performed. The invention permits use of automation and implementation of the assay in a 1536 well format. A homogenous assay also allows the use of non-adherent cells. The assays described herein will permit ultra high throughput screening, i.e, screening in plates containing more than 384 wells, of compounds with uncoupling activity in mitochondria. In addition, the assay window is superior to previous methods which improves its utility in ultra high throughput screening experiments. In particular, variation between replicates is improved due to the removal of wash steps which can damage and/or dislodge cells from the substrate.

The present invention is based, in part, on the discovery that correcting for inner filter effects using a compound, such as Brilliant Black BN, to quench fluorescence, it is possible to detect changes in mitochondrial membrane potential using high-throughput, homogeneous assay format in vertebrate cells. It has been found that without the use of Brilliant Black BN, a washing step to remove unbound fluorescent dye is required.

Briefly, the methods of the invention involve the following steps. First, vertebrate cells are incubated in an assay buffer. Test compounds are added that potentially will modulate the physiology of the mitochondrion. These agents could be chemical uncouplers, mitochondrial poisons, uncoupling proteins, ionophores, etc. Following incubation with the test compound, a fluorescent dye whose fluorescence is a function of the mitochondrial membrane potential in the cell, e.g., tetramethylrhodamine methyl ester (TMRM), is added to the cells. Without being bound by any theory, it is believed that the fluorescent dye is attracted to the mitochondria in proportion to the mitochondrial membrane potential. At higher potential, more dye accumulates and is contained in the mitochondrion. Following incubation, an inner filter, such as Brilliant Black BN, is added to the cells to quench non-specific fluorescence. The effect of the test compound on the fluorescence of the intra-mitochondrial fluorescent dye, e.g, TMRM, is then measured. The effect of the test compound may be compared to a negative control, i.e., where no test compound is added, and a positive control, i.e., to a known mitochondrial uncoupler such as 2,4-dinitrophenol (DNP). As noted above, no removal of solution or washing steps are performed. Also, the test compound can be added either before or after addition of the fluorescent dye. The positive and/or negative control may be performed at the same time or at different times than the test compound.

In certain embodiments, the cell can express an uncoupling protein, e.g., by recombinant means. In such embodiments, an ability of the test agent to modulate uncoupling activity in the presence of the uncoupling protein, but not in the absence of the uncoupling protein, indicates that the modulatory activity of the agent is specific for the uncoupling protein.

The present invention is believed to be useful for the identification of activators or inhibitors of mitochondrial uncoupling proteins and/or uncoupling activity, identification of chemical uncouplers (uncoupling protein-independent, uncoupling activity), characterization of compounds to identify those with mitochondrial toxicity, and identification of compounds stimulating mitochondial biogenesis. Such compounds may be useful in the treatment of diseases or conditions associated with uncoupling activity. For example, compounds that are capable of modulating uncoupling activity, either intrinsically or by modulating uncoupling protein activity, may be useful in the treatment of metabolic or weight disorders such as obesity. Compounds capable of modulating uncoupling are also useful in the study of the mechanisms, causes, and consequences of uncoupling in cells, for example by identifying proteins or other compounds that the compound associates with in vivo or in vitro, or by creating animal models of uncoupling related metabolic or weight disorders. Such animal models are useful, e.g., for the study of such disorders, as well as for the identification of compounds useful in the treatment or prevention of the disorders. As used herein, “compound” is not limited to a chemical compound, but refers to any molecule that may have biological activity including, but not limited to, peptides, nucleic acids, carbohydrates, lipids, antibodies, or any other organic or inorganic molecule.

Known activators of mitochondrial uncoupling proteins include retinoids (see Rial et al., 1999, EMBO J 18:5827-33; Tomas et al., 2004, Biochim Biophys Acta. 1658:157-64), fatty acids such as phytanic acid (see Schluter et al., 2002, Biochem J 362:61-9) and nitric oxide (see Nisoli et al., 2004, Proc Natl Acad Sci USA 101:16507-12).

DNP is the best known chemical uncoupler; but many other compounds are known to induce uncoupling. DNP derivatives such as 4,6-dinitro-o-cresol (Victoria Yellow) and 2,4-dinitro-1-naphtol (Martius Yellow) as well as structurally unrelated compounds such as 2,6-di-t-butyl-4-(2′,2′-dicyanovinyl)phenol) (SF6847) (also known as 2-(3,5-di-tert-butyl-4-hydroxybenzylidene)-malononitrile), carbonylcyanide m-chlorophenylhydrazone (CCCP) and carbonylcyanide-p-trifluoromethoxy-phenylhydrazone (FCCP) (Miyoshi et al., 1987, Biochim Biophys Acta 891:293-299) are uncouplers. Another class of chemical uncouplers is the salicylanilides of which S-13 is the most potent compound discovered so far (Terada et al., 1988, Biochim Biophys Acta 936:504-512).

Examples of mitochondrial poisons include 2-deoxy-D-ribose, potassium cyanide, and rotenone (see Zamzami et al., 1995, J. Exp. Med. 181:1661-1672).

The present methods are also useful in the identification of compounds that modulate cellular processes associated with changes in mitochondrial membrane potential, such as apoptosis. In such embodiments, a cell is typically contacted with a test compound in the presence of an apoptosis-inducing compound or treatment, and the ability of the test compound to modulate the uncoupling associated with the compound or treatment is assessed. Test compound that are found to be capable of modulating apoptosis-associated uncoupling are useful in the study of apoptosis, as well as in the treatment of any of a large number of diseases and conditions associated with apoptosis, including, but not limited to, inflammatory diseases, viral infections, neurodegenerative diseases, cancers and heart disease, or as a method of inducing apoptosis in undesired cells in vivo.

Reference to open-ended terms such as “comprises” allows for additional elements or steps. Occasionally phrases such as “one or more” are used with or without open-ended terms to highlight the possibility of additional elements or steps.

Unless explicitly stated reference to terms such as “a” or “an” is not limited to one. For example, “a cell” does not exclude “cells”. Occasionally phrases such as one or more are used to highlight the possible presence of a plurality.

### Assay and Assay Components

Assay

An exemplary protocol is provided below. A person of skill in the art can readily modify this protocol based on the teachings and disclosure found herein. All cell numbers, volumes, times and concentrations described below can be adjusted by ±5%, ±10%, ±20%, 30%, ±40%, f 50%, or greater. All temperatures described below can be adjusted by ±1° C., ±2° C., ±3° C., ±4° C., or ±5° C. or greater.

Vertebrate cells are plated manually, or by an automated system such as SelecT™ (The Automation Partnership, Wilmington, Del.), the day before the assay at 100,000 cells/well in 100 μl of a culture media suitable for growth of the cells for 96-well plates or 75,000 cells/well in 50 μl of a culture media suitable for growth of the cells for 384-well plates (BD Optilux Cat. No. 353220, BD Biosciences, San Jose, Calif.) or 10,000 cells per well of a culture media suitable for growth of the cells and maintained at 37° C./5% CO₂. On the day of the assay, before compound addition, assay buffer (50 mM Hepes, pH 7.4 (Invitrogen Corporation, Carlsbad, Calif., Cat. No. 15630), 150 mM KCL, 160 mM NaCl, 50 mM D-(+)-Glucose Solution (Sigma-Aldrich, St. Louis, Mo., Cat. No. G8769) is added at a volume equal to that of culture medium used for cell seeding. Cells plated may be adherent or non-adherent cells.

The test compound in 100% DMSO or diluted into 50% DMSO/50% assay buffer is added to the cells (usually 2 μl compound in the 96 well format, 1 μl in the 384 well format or 80 nl in the 1536 well format). The cells are incubated with the test compound at 37° C./5% CO₂ for 30 minutes.

An appropriate amount, for example, 20 μl for a 96-well plate, of 16.5 μM stock of a fluorescent dye such as TMRM (in 10% DMSO) is added to a final concentration of 150 nM in 1% DMSO. The cells are incubated for 20 minutes at room temperature.

An appropriate amount of Brilliant Black BN stock made in D-PBS is added to have a 1.6 mM final concentration of Brilliant Black BN. In situations in which non-adherent cells are assayed, cells are collected on the culture plate surface by centrifugation at 500×g for 1 minute at room temperature.

The fluorescence is immediately read on a fluorescent detector such as a Molecular Devices Analyst HT (Ex=530 nm, Em=580 nm, 561 nm filter).

Cells

Any vertebrate cell type can be used in the present invention. In particular, any mammalian cell can be used. Any such cell type can be used, including primary cell lines, secondary cell lines, transformed cells, and others, and including whole (untreated) cells and permeabilized cells. A number of cell types are described by the American Type Culture Collection (ATCC, Manassas, Va.) or in Freshney, 1994, Culture of Animal Cells: A Manual of Basic Technique, Wiley-Liss, New York, any of which can be used. For example, CHO, HEK-293, HepG2, Hepa1C1C7, C2C12, CV-1, HeT, murine myelomas, n51, SF9, VERO, and other cells can be used. In some embodiments, a vertebrate cell that normally expresses a UCP protein can be used. For example, a brown adipose cell expressing UCP1 can be used, or a brain, muscle, or fat cell expressing UCP2 can be used.

The culture of cells used in the assays of the present invention, including cell lines and cultured cells from tissue or blood samples is well known in the art. Freshney (Culture of Animal Cells, a Manual of Basic Technique (3^{rd} ed. 1994)) and the references cited therein provides a general guide to the culture of cells. Additional information on cell culture is found in Ausubel and Sambrook, supra. Cell culture media are described in The Handbook of Microbiological Media (Atlas & Parks, eds., 1993). Additional information is found in commercial literature such as the Life Science Research Cell Culture Catalogue (1998) from Sigma-Aldrich (St. Louis, Mo.). Cells can be grown in bulk flasks and added to the substrate (e.g., microtiter plate) or can be grown directly on the substrate (e.g., in the wells of the microliter plate, depending on the intended application and available equipment.

Cells can be used at any of a wide range of densities, depending on the dye, the test agent, and the particular assay conditions. Preferably, a density of about OD₆₀₀=0.01 to 1 is used, more preferably between about 0.05 and 0.5, most preferably about 0.1.

Cells can be seeded at any of a wide range of numbers, depending on the dye, the test agent, and the particular assay conditions. Preferably, for 96-well plates, about 50,000 to 150,000 cells are used in a single well. Preferably, for 384-well plates, about 10,000 to 100,000 cells are used in a single well. Preferably, for 1536-well plates, about 5,000 to 12,000 cells are used in a single well.

Uncoupling Proteins

A large number of uncoupling proteins have been identified from numerous organisms, any of which can be used in the present invention. For example, UCP1, UCP2 (see, e.g., Fleury, et al., 1997, Nature Genetics 15:269), UCP3, UCP4 (see, e.g., Mao et al., 1999, FEBS Lett., 443:326), BMCP1 (see, e.g., Sanchis et al., 1998, J. Biol. Chem. 273:34611) or homologs or derivatives thereof, can be used. UCPs have been shown to possess proton transporting activity, and to typically have six alpha-helical transmembrane domains. UCP1-4 are homologous to each other. UCP proteins suitable for the present invention can be derived from any vertebrates. In preferred embodiments, a mammalian UCP sequence, preferably a mouse or human UCP sequence, is used. Amino acid and nucleotide sequences for a multitude of UCP proteins can be found, e.g., by accessing GenBank at the National Institute of Biotechnology Information (see, e.g. accession numbers Y18291, NM_{—}003356.1, AF096289, AF110532, AF036757, AF092048, and others). UCP proteins are also described, e.g., in U.S. Pat. No. 5,853,975.

In certain embodiments, a hybrid form of an uncoupling protein can be used. Such hybrid forms can include a UCP protein, or fragment thereof, as well as a heterologous polypeptide sequence such as a label, antigenic sequence, or, preferably, a leader sequence that facilitates the insertion of the protein into the mitochondrial membrane.

Expressing Uncoupling Proteins in Cells

In certain embodiments, one or more UCP proteins will be expressed in vertebrate cells. Methods for expressing heterologous proteins in cells are well known to those of skill in the art, and are described, e.g., in Ausubel et al. (2007, Current Protocols in Molecular Biology, Wiley Interscience), Freshney, 1994, Culture of Animal Cells: A Manual of Basic Technique, Wiley-Liss, New York, and others. Typically, in such embodiments, a polynucleotide encoding a UCP protein will be operably linked to an appropriate expression control sequence for the particular host cell in which the UCP protein is to be expressed. Promoters and other elements for expressing heterologous proteins in vertebrate cells are commonly used and are well known to those of skill. See, e.g., Cruz & Patterson (1973) Tissue Culture, Academic Press; Meth. Enzymology 68 (1979), Academic Press; Freshney, 3rd Edition (1994) Culture of Animal Cells: A Manual of Basic Techniques, Wiley-Liss.

Promoters and control sequences for such cells include, e.g., the commonly used early and late promoters from Simian Virus 40 (SV40), or other viral promoters such as those from polyoma, adenovirus 2, bovine papilloma virus, or avian sarcoma viruses, herpes virus family (e.g., cytomegalovirus, herpes simplex virus, or Epstein-Barr Virus), or immunoglobulin promoters and heat shock promoters (see, e.g. Sambrook et al. (2001, Molecular Cloning: A Laboratory Manual (Third Edition), Cold Spring Harbor Laboratory Press; Ausubel et al. (2007, Current Protocols in Molecular Biology, Wiley Interscience), Meth. Enzymology (1979, 1983, 1987), Pouwells, et al., supra (1987)). In addition, regulated promoters, such as metallothionein, (i.e., MT-1 and MT-2), glucocorticoid, or antibiotic gene “switches” can be used. Enhancer regions of such promoters can also be used.

Expression cassettes are typically introduced into a vector that facilitates entry of the expression cassette into a host cell and maintenance of the expression cassette in the host cell.

Such vectors are commonly used and are well know to those of skill in the art. Numerous such vectors are commercially available, e.g., from Invitrogen, Stratagene, Clontech, etc., and are described in numerous guides, such as Ausubel et al., 2007, Current Protocols in Molecular Biology, Wiley Interscience. Such vectors typically include promoters, polyadenylation signals, etc. in conjunction with multiple cloning sites, as well as additional elements such as origins of replication, selectable marker genes (e.g., LEU2, URA3, TRP1, HIS3, GFP), centromeric sequences, etc. Any of a number of vectors can be used, such as pSV2, pBC12BI, and p91023, as well as lytic virus vectors (e.g., vaccinia virus, adenovirus, baculovirus), episomal virus vectors (e.g., bovine papillomavirus), and retroviral vectors (e.g., murine retroviruses).

Standard transfection methods are used to produce vertebrate cell lines that express the nucleic acid. Transformation of eukaryotic cells are performed according to standard techniques (see, e.g., Clark-Curtiss & Curtiss, Methods in Enzymology 101:347 362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra).

Fluorescent Dyes/Assay Buffer

The present invention can be practiced using any dye whose distribution, intensity, or spectral characteristics change with changing mitochondrial membrane potential. Such dyes enter the cell and are accumulated in the mitochondrion. For example, any probe listed in the chapter on potentiometric probes in the Molecular Probes (Eugene, Ore.) catalog can be used. Such probes include fast response probes (including styryl and hybrid oxonol probes) and, preferably, slow response probes, including, but not limited to, carbocyanine probes (e.g., DiOC₂(3), DiOC₂(5), DiOC₅(3), DiOC₇(3), DiSC₂(5), DiSC₃(5), DiIC₁(3), and JC-1), rhodamine probes (e.g., tetramethylrhodamine methyl and ethyl esters, rhodamine 123), oxonol probes (e.g., oxonol V, oxonol VI, DiBAC₄(3), DiBAC₄(5), and DiSBAC₂(3)), merocyanine probes (e.g., merocyanine 540), and tetramethylrosamine chloride. In addition, O-safranine can be used to monitor mitochondrial membrane potential by measuring the absorption of visible light (OD₅₃₀ nm-OD₄₉₀ nm). The suitability of any of these described probes in the present assays can readily be assessed, e.g., by contacting one or more cells in a homogeneous format with the dye and with a compound that is known to alter the mitochondrial membrane potential (e.g., FCCP or CCCP), and detecting the fluorescence in the sample. Any dye that allows the detection of a change in fluorescence under such conditions can be used in the present assays. In preferred embodiments, tetramethylrhodamine methyl (TMRM) is used.

Such dyes can be added at any concentration that allows detection using standard methodology. Preferably, a concentration of between about 0.05 μM and about 1 μM is used, more preferably between about 0.05 μM and about 0.5 μM and, most preferably, about 0.1 μM. Typically, such dyes will be added to cells for between about 5 and about 30 minutes. TMRM must be dissolved first in 100% DMSO, and then further diluted into a mixture of 80% water/20% DMSO prior to adding to the cells. Usually 1/10^{th} volume of this TMRM solution is added to the cells.

Inner Filter Effects

The use of a compound that reduces the inner filter effects is critical to the performance of invention. The compound is added after incubating the cells with a fluorescent dye to quench non-specific fluorescence. Without being bound by any mechanism, the inner filter does not enter the cell, but remains in the bulk solution, quenching “non-specific” fluorescence.

In assays known in the art, a washing step is required to remove free compounds and probe. See Huang, 2002, J Biomolecular Screening 7:383-389. The use of the compound that reduces the inner filter effects obviates the need for a washing step permitting ultra high throughput screening. Preferred inner filters include, but are not limited to Brilliant Black BN (Sigma-Aldrich, St. Louis, Mo.). A particular inner filter is chosen based upon its physical properties in order for its absorption peak to match the emission of the chosen dye. Representative inner filters for exemplary dyes are shown in Table 1 and are available from commercial suppliers such as Sigma-Aldrich. The concentration of inner filter that can be used can range from 0.5 mM to 6 mM, preferably 1 mM to 2.5 mM, in, for example, phosphate buffered saline. Suitable concentrations include 0.5 mM, 1.0 mM, 1.5 mM, 1.6 mM, 2.0 mM, 2.5 mM, and 3.0 mM. The optimal concentration of Brilliant Black BN was found to be 1.6 mM.

The inner filter quenches the non-specific fluorescence immediately so that the plates must be read immediately, i.e, within 30 seconds, 1 minutes, or up to 5 minutes, after addition of the inner filter.

TABLE 1
Mitochondrial Potential Probes and Inner Filters

| | | Inner Filter 1 | |
|---|---|---|---|
| Probe | Emission (nm) | (most preferred) | Inner Filter 2 (preferred) |
| TMRM | 573 | Brilliant Black BN | Chicago Sky Blue 6B |
| TMRE | 574 | Brilliant Black BN | Chicago Sky Blue 6B |
| Rhodamine 123 | 529 | Acid Fuchsin | |
| Tetramethylrosamine | 574 | Brilliant Black BN | Chicago Sky Blue 6B |
| chloride | | | |

Test Agents

Essentially any chemical compound can be used as a potential activity modulator in the assays of the invention, although most often compounds that can be dissolved in aqueous or organic (especially DMSO-based) solutions are used. The assays can be designed to screen large chemical libraries by automating the assay steps and providing compounds from any convenient source to assay, which are typically run in parallel (e.g., in microtiter formats on microtiter plates in robotic assays). It will be appreciated by those of skill in the art that there are many commercial suppliers of chemical compounds, including Sigma-Aldrich (St. Louis, Mo.), Fluka Chemika-Biochemica Analytika (Buchs Switzerland), and the like.

Suitable controls include well known chemical uncouplers such as DNP, carbonyl cyanide-p-trifluoromethoxyphenylhydrazone (FCCP) and carbonyl cyanide 3-chlorophenylhydrazone (CCCP).

In one preferred embodiment, high-throughput screening methods involve providing a combinatorial library containing a large number of potential therapeutic compounds (potential modulator compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (e.g., amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, 1991, Int. J. Pept. Prot. Res.; 37:487-493, and Houghton et al., 1991, Nature 354:84-88). Other chemistries for generating chemical diversity libraries can also be used. Such chemistries include, but are not limited to, peptoids (International Publication No. WO 91/19735); encoded peptides (International Publication No. WO 93/20242); random bio-oligomers (International Publication No. WO 92/00091); benzodiazepines (U.S. Pat. No. 5,288,514); diversomers, such as hydantoins, benzodiazepines and dipeptides (Hobbs et al., 1993, Proc. Nat. Acad. Sci. USA 90:6909-6913); vinylogous polypeptides (Hagihara et al., 1992, J. Amer. Chem. Soc. 114:6568); nonpeptidal peptidomimetics with β-D-glucose scaffolding (Hirschmann et al., 1992, J. Amer. Chem. Soc. 114:9217-9218); analogous organic syntheses of small compound libraries (Chen et al., 1994, J. Amer. Chem. Soc. 116:2661); oligocarbamates (Cho et al., 1993, Science 261:1303); and/or peptidyl phosphonates (Campbell et al., 1994, J. Org. Chem. 59:658); nucleic acid libraries; peptide nucleic acid libraries (see, e.g., U.S. Pat. No. 5,539,083); antibody libraries (see, e.g., Vaughn et al., 1996, Nature Biotechnology 14:309-314, and International Publication No. WO9700271); carbohydrate libraries (see, e.g., Liang et al., 1996, Science 274:1520-1522 and U.S. Pat. No. 5,593,853); small organic molecule libraries (see, e.g., benzodiazepines, Baum, 1993, C&E News, Jan. 18, 1993, page 33; isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J.; Asinex, Moscow, Russia; Tripos, Inc., St. Louis, Mo.; ChemStar, Ltd., Moscow, Russia; 3D Pharmaceuticals, Exton, Pa.; Martek Biosciences, Columbia, Md., etc.).

### Fluorescence Detection

A fluorometer that measures fluorescent energy is used to detect the fluorescent signal. A fluorometer may be anything from a relatively simple, manually operated instrument that accommodates only a few sample tubes at a time, to a somewhat more complex manually operated or robotic instrument that accommodates a larger number of samples in a format such as, e.g., a 96-, 384- or 1536-well microplate (such as, e.g., an fmax™ fluorimetric plate reader, Analyst GT, or Analyst HT, or Tetra, Molecular Devices Corp., Sunnyvale, Calif.; or a Cytofluor fluorimetric plate reader, model #2350, Millipore Corp., Bedford, Mass.), or a complex robotic instrument (such as, e.g., a FLIPR™ instrument; Molecular Devices, Sunnyvale, Calif.) that accommodates a multitude of samples in a variety of formats such as 96-well microplates. When high throughput (HTS) assaying of a large number of samples is desired, robotic or semi-robotic instruments are preferred. Excitation sources may be lamps, lasers, LEDs, etc.

Preferably, the fluorimeter measures the plates or samples from the bottom so as not to excite and measurement from the bottom in order to minimize quenching of specific fluorescence by the inner filter in the bulk solution.

### High-Throughput Format

In preferred embodiments, the invention provides in vitro assays for uncoupling activity in an ultra high-throughput format. Control reactions that measure uncoupling activity in a reaction that does not include an uncoupling activity modulator are optional, as the assays are highly uniform. However, such optional control reactions can be appropriate and increase the reliability of the assay. Accordingly, in a preferred embodiment, the methods of the invention include such a control reaction.

In some assays, it will be desirable to have positive controls to ensure that the components of the assays are working properly. For example, a known activator of uncoupling activity can be incubated with one sample of the assay, and the resulting increase in uncoupling activity determined according to the methods herein. In preferred embodiments, CCCP, or carbonyl-cyanide p-chlorophenylhydrazone, is used. CCCP can be added at any concentration sufficient to effect a detectable amount of uncoupling. For example, 0.1 μM, 1 μM, 10 μM, 100 μM or 1 mM can be used. In preferred embodiments, 0.1 to 5 μM can be used and, more preferably, 2 μM is used. CCCP is readily available from commercial sources, e.g., Sigma-Aldrich. See, e.g., Heytler et al., 1962, Biochem Biophys Res Commun 7:272.

In embodiments where an uncoupling protein is expressed in a cell, a known modulator of the uncoupling protein is preferably used. For example, the commercially available UCP1 activator 2-bromo-palmitate (BrPalm) can be used (at, e.g., 5 to 10 μM), thereby providing a UCP1-specific increase in uncoupling activity. In addition, a known inhibitor of UCP activity can be added, and the resulting decrease in uncoupling activity similarly detected. For example, GDP can be used to inhibit UCP1, at, e.g., 100 μM.

In the high-throughput assays of the invention, it is possible to screen ten to twenty thousand or more different compounds in a single day. In particular, each well of a microtiter plate can be used to run a separate assay against a selected potential modulator, or if concentration or incubation time effects are to be observed, every 5-10 wells can test a single modulator. Most commonly, the assay will use 96, 384 or 1536 well microtiter plates. The reactions are preferably performed in a standard microtiter plate with 1536 sample wells. Microplates with 3456 or even 9600 wells can be used. It is possible to assay many different plates per day; assay screens for up to about 100,000-1,000,000 different compounds are possible using the integrated systems of the invention.

### Integrated Systems

The invention also provides integrated systems for high-throughput screening of potential modulators of uncoupling activity. Such systems typically include a robotic armature which transfers fluid from a source to a destination, a controller which controls the robotic armature, a label detector, a data storage unit which records label detection, and an assay component such as a microtiter dish.

A number of well-known robotic systems have also been developed for solution phase chemistries. These systems include automated workstations like the automated synthesis apparatus developed by Takeda Chemical Industries, LTD (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art.

Any of the assays for compounds that modulate uncoupling activity, as described herein, are amenable to high-throughput screening. High-throughput screening systems are commercially available (see, e.g., Zymark Corp. (Hopkinton, Mass.); Air Technical Industries (Mentor, Ohio); Beckman Instruments, Inc. (Fullerton, Calif.); Precision Systems, Inc., (Natick, Mass.), etc.). Such systems typically automate entire procedures including all sample and reagent pipetting, liquid dispensing, timed incubations, and final readings of the microplate in detector(s) appropriate for the assay. These configurable systems provide high-throughput and rapid start up as well as a high degree of flexibility and customization. The manufacturers of such systems provide detailed protocols for the various high-throughput systems.

### Identification of Compounds Affecting Mitochondrial Uncoupling

It will be appreciated that the methods provided herein can be used to screen any test agent for its ability to alter mitochondrial membrane potential in a vertebrate cell. For example, these methods can be used for screening for activators of mitochondrial uncoupling proteins, chemical uncouplers, compounds with mitochondrial toxicity, and compounds which stimulate mitochondrial biogenesis.

Uncoupling of mitochondrial oxidative phosphorylation is a potential target for the treatment of obesity since it is thought to mimic the increased metabolic rate seen with exercise. See Harper et al., 2001 Obesity Rev 2:255-265. Decreasing the mitochondrial membrane potential may also by a therapeutic strategy for diabetes (see Green et al., 2004, Diabetes 53:S110-S118); and acute central nervous system injury resulting from stroke or trauma (see Maragos et al., 2004, J Neurochem 91:257-262; Mattiasson et al., 2003, Nat Med 9:1062-1068).

Thus, in addition to molecules with intrinsic uncoupling activity, or that modulate uncoupling proteins, these methods can be used to identify agents that indirectly affect mitochondrial membrane potential. For example, molecules that induce apoptosis, which is characterized by a loss of mitochondrial membrane potential, can be screened. In a typical embodiment, cells are exposed to a ΔΨm-dependent fluorescent probe, contacted with a test agent, and the fluorescence is detected. Apoptosis is thought to be involved in neurodegenerative diseases such as Alzheimer's disease and Parkinson's disease (see Talton et al., 1998, Ann. Neurol. 44:S134-S141).

Preferably, such methods are performed in a high-throughput format, allowing the rapid and efficient screening of a large number of test agents.

Methods for screening of compounds with mitochondrial toxicity can be used during preclinical safety evaluation. See Amacher, 2005, Curr Med Chem 12:1829-1839.

Test agents identified as “hits” in such screens can be subject to any appropriate secondary screening step known to one of skill in the art. Such screens could include measurement of oxygen consumption using different energetic substrates, NADH oxidation, permeability transition, mitochondrial calcium accumulation, ATP synthesis, cytochrome c oxidase activity, or citrate synthase activity.

Potential hits can also be further tested in animal studies according to methods known to one of skill in the art. Such screens could include measurement of mitochondria complex I, II, DI or IV mass isolated from tissues of treated animals using immunoassays or enzymatic activity of complex I, IV, or pyruvate dehydrogenase from tissues of treated animals using enzymatic assays using commercially available products (Mitosciences Inc., Eugene, Oreg.).

The specific embodiments described herein are offered by way of example only, and the invention is to be limited only by the terms of the appended claims along with the full scope of equivalents to which such claims are entitled. Indeed various modifications of the invention, in addition to those shown and described herein will become apparent to those skilled in the art from the foregoing description and accompanying drawings. Such modifications are intended to fall within the scope of the appended claims.

All references cited herein, including patent applications, patents, and other publications, are incorporated by reference herein in their entireties for all purposes.

### Examples

### Example 1

In order to determine if an inner filter would permit omission of wash steps used in a TMRM-based mitochondrial membrane potential assay, the dye Brilliant Black BN was used as a potential inner filter. Mitochondrial activity was assessed using the uncoupling agents 2,4-DNP and CCCP.

24 hours before assay, CHO—K1 cells were seeded in a 384 well plate at 75,000 cells per well in 50 μl growth medium (Iscove's Modified Dulbecco's Medium Invitrogen, 12440-046, 10% Fetal Bovine Serum (Invitrogen, 26140-095), 1× HT Supplement (Invitrogen, 11067-030) 1× Penicillin-Streptomycin solution (Hyclone, SV30010) 2 mM Glutamine (Hyclone, SH30034.01). Cells were then incubated overnight at 37° C., 5% carbon dioxide. When commencing the assay, 50 μl of assay buffer (50 mM Hepes, pH 7.4 150 mM KCL, 1600 mM NaCl, 25 mM D-(+)-glucose) was added to cells. 1 μl of the various amounts of the mitochondrial chemical uncouplers 2,4-DNP or FCCP was added and the cells are then incubated for 30 minutes at 37° C., 5% carbon dioxide. 10 μl of 9 μM fluorescent dye TMRM (in 10% DMSO) was added and cells are incubated for 20 minutes at room temperature. 10 μl of 16 mM Brilliant Black BN prepared in D-PBS was added to yield a 1.6 mM final concentration of Brilliant Black BN. The fluorescence was immediately read on a Molecular Devices Analyst HT fluorescent plate reader, using an excitation wavelength of 530 nM and measuring the emission at 580 nm.

This method using Brilliant Black BN as an inner filter successfully measured mitochondrial membrane potential. Membrane potential was observed to decrease with increasing amounts of the uncouplers. The EC50 of 2,4-DNP was 5 μM and that of CCCP was 250 nM, in keeping with established values.

### Example 2

In order to determine a range of measurement using the homogenous membrane potential assay, an experiment was performed using the cation ionophore, nigericin, known to hyperpolarize mitochondria due to its H+—K+ exchange activity between the mitochondrial matrix and inner mitochondrial membrane.

24 hours before assay, CHO—K1 cells were seeded in a 384 well plate at 75,000 cells per well in 50 μl growth medium (Iscove's Modified Dulbecco's Medium, lnvitrogen, 12440-046, 10% Fetal Bovine Serum (Invitrogen, 26140-095), 1× HT Supplement (Invitrogen, 11067-030) 1× Penicillin-Streptomycin solution (Hyclone, SV30010) 2 mM Glutamine (Hyclone, SH30034.01). Cells were then incubated overnight at 37° C., 5% carbon dioxide. When commencing the assay, 50 μl of assay buffer (50 mM Hepes, pH 7.4 150 mM KCL, 1600 mM NaCl, 25 mM D-(+)-glucose) was added to cells. 1 μl of the various amounts of the cation ionophore, H+—K+ antiporter, nigericin, was added and the cells are then incubated for 30 minutes at 37° C., 5% carbon dioxide. 10 μl of 9 μM fluorescent dye TMRM (in 10% DMSO) was added and cells are incubated for 20 minutes at room temperature. 10 μl of 16 mM Brilliant Black BN prepared in D-PBS was added yield a 1.6 mM final concentration of Brilliant Black BN. The fluorescence was immediately read on a Molecular Devices Analyst HT fluorescent plate reader, using an excitation wavelength of 530 nm and measuring the emission at 580 nm.

Nigericin caused hyperpolarization of the mitochondrial membrane which increased until greater levels of nigericin caused toxicity and membrane depolarization. These data indicate the homogenous assay can measure both hyperpolarization and depolarization.

### Example 3

In order to determine if the homogenous assay can measure membrane potential in dissociated adherent cells or cells which do not grow adhering to substrate, the homogeneous assay was performed using dissociated cells. At time of assay, CHO—K1 cells were dissociated from growth substrate by washing cells with phosphate buffered saline and the incubating the cells in enzyme-free cell dissociation buffer (Speciality Media) at 30 μl solution per cm² surface area. Cells were resuspended in Suspension Assay Buffer (25 mM HEPES, pH 7.4, 75 mM KCl, 80 mM NaCl, 25 mM D-glucose). 100,000 cells in 100 μl of suspension assay buffer were placed in wells of a 384-well plate. 1 μl of the various amounts of the mitochondrial uncoupling compound, CCCP, were added and the cells are then incubated for 30 minutes at 37° C., 5% carbon dioxide. 10 μl of 9 μM fluorescent dye TMRM (in 10% DMSO) was added and cells are incubated for 20 minutes at room temperature. 10 μl of 16 mM Brilliant Black BN prepared in D-PBS was added yield a 1.6 mM final concentration of Brilliant Black BN. Cells were collected at the bottom surface of the assay plate by centrifugation at 500×g for 1 minute. The fluorescence was immediately measured on a Molecular Devices Analyst HT fluorescent plate reader, using an excitation wavelength of 530 nm and measuring the emission at 580 nm.

The homogenous assay was able to measure mitochondrial membrane potential in dissociated cells. The potency of CCCP was similar to that observed in the adherent assay.

### Example 4

In order to determine whether the homogeneous assay was capable of measuring mitochondrial membrane potential in a miniaturized ultra high-throughput format, mitochondrial potential was measured in cells seeded in 1536 well plates.

24 hours before assay, CHO—K1 cells were seeded in a 1536 well plate at 10,000 cells per well in 4 μl growth medium (Iscove's Modified Dulbecco's Medium Invitrogen, 12440-046, 10% Fetal Bovine Serum (Invitrogen, 26140-095), 1×HT Supplement (Invitrogen, 11067-030) 1× Penicillin-Streptomycin solution (Hyclone, SV30010) 2 mM Glutamine (Hyclone, SH30034.01). Cells were then incubated overnight at 37° C., 5% carbon dioxide. When commencing the assay, 4 μl of assay buffer (50 mM Hepes, pH 7.4 150 mM KCL, 1600 mM NaCl, 25 mM D-(+)-glucose) was added to cells. 80 nl of various amounts of the mitochondrial uncoupling compound, CCCP, was added and the cells were then incubated for 30 minutes at 37° C., 5% carbon dioxide. 0.8 μl of 6 μM fluorescent dye TMRM (in 20% DMSO) was added and cells were incubated for 20 minutes at room temperature. 0.8 μl of 16 mM Brilliant Black BN prepared in D-PBS was added yield a 1.6 mM final concentration of Brilliant Black BN. The fluorescence was immediately read on a Molecular Devices Tetra fluorescent plate reader, using an excitation wavelength of 530 nm and measuring the emission at 580 nm while employing a custom filter (Omega Optical) whose transmission wavelength was 580 nm±10 nm.

The method using Brilliant Black BN successfully measured mitochondrial membrane potential in the miniaturized format. The EC50 of CCCP was 180 nM, in keeping with established values.

What is claimed is:

1. A method for screening a test compound for the ability to modulate mitochondrial membrane potential within a vertebrate cell comprising the steps of:
a) incubating said vertebrate cell in the presence of said test compound;
b) adding a fluorescent dye into said vertebrate cell, wherein the fluorescence of said cell in the presence of said fluorescent dye is a function of the mitochondrial membrane potential in said cell;
c) adding an inner filter to said vertebrate cell to quench non-specific fluorescence; and
d) comparing the fluorescence in said cell to the fluorescence of a cell incubated in the absence of said test compound;
wherein an alteration in the fluorescence in said cell in the presence of said test compound compared to the fluorescence in the absence of said test compound indicates an ability of said test compound to modulate mitochondrial membrane potential; and
wherein said screening is performed in a homogeneous format.

2. The method of claim 1, wherein said vertebrate cell is a mammalian cell.

3. The method of claim 1, wherein said fluorescent dye is selected from the group consisting of tetramethylrhodamine, methyl ester (TMRM) and tetramethylrhodamine, ethyl ester (TMRE).

4. The method of claim 1, wherein said inner filter is Brilliant Black BN.

5. The method of claim 1, further comprising recombinantly expressing an uncoupling protein in said vertebrate cell.

6. The method of claim 5, wherein said uncoupling protein is selected from the group consisting of UCP1, UCP2, UCP3, UCP4, or UCP5.

7. The method of claim 1, wherein said measuring is performed using a multi-well plate.

8. The method of claim 7, wherein said multi-well plate is a 384- or 1536-well plate.

9. The method of claim 8, wherein a plurality of test compounds is screened in parallel with each other.

10. The method of claim 1, wherein said measuring is performed in a high-throughput format

11. The method of claim 10, wherein said high-throughput format comprises an automatic pipetting station, a robotic armature, and a robotic controller.

12. The method of claim 1, wherein the test compound is an activator of a mitochondrial uncoupling protein.

13. The method of claim 12, wherein the mitochondrial uncoupling protein is selected from the group consisting of UCP1, UCP2, UCP3, UCP4, or UCP5.

14. The method of claim 1, wherein the test compound is a chemical uncoupler.

15. The method of claim 1, wherein the test compound has mitochondrial toxicity.

16. The method of claim 1, wherein the test compound stimulates mitochondrial biogenesis.

17. The method of claim 1, wherein the fluorescence of said cell incubated in the presence of said test compound is detected at the same time as the fluorescence of said cell incubated in the absence of said test compound is detected.

18. The method of claim 1, wherein the fluorescence of said cell incubated in the presence of said test compound is detected at a different time than when the fluorescence of said cell incubated in the absence of said test compound is detected.
